(19) United States
(12) Patent Application Publication
(10) Pub. No.: US 2022/0088956 A1
(43) Pub. Date: Mar. 24, 2022

(54) METHOD OF MANUFACTURING A SANITISED PAPER ARTICLE

(71) Applicant: TRIMFOLD ENVELOPES LIMITED, Co. Meath (IE)

(72) Inventor: Eugene Healy, Co. Meath (IE)

(73) Assignee: TRIMFOLD ENVELOPES LIMITED, Co. Meath (IE)

(21) Appl. No.: 17/476,580

(22) Filed: Sep. 16, 2021

(30) Foreign Application Priority Data
Sep. 18, 2020 (GB) 2014763.3

(51) Int. Cl. B41M 7/00; B41M 1/04; B41M 5/50

(52) U.S. Cl. CPC B41M 7/0045; B41M 5/502; B41M 1/04

(57) ABSTRACT

A method of manufacturing a sanitised paper article 202 comprising the steps: supplying a paper sheet 12 having a first surface and a second surface to a flexographic printing apparatus 2 having a first sanitising module 10a, applying a sanitising solution to at least the first surface of the paper sheet 12 using the first sanitising module 10a, and forming an article from the sanitised paper sheet 12.

### TECHNICAL FIELD

The present invention relates to a method of manufacturing a sanitised paper article and particularly, although not exclusively, relates to a method of manufacturing a sanitised paper article, such as an envelope, using a flexographic printing apparatus.

### BACKGROUND

Cutlery envelopes are used in the catering industry in order to pre-package individual sets of cutlery for customers. For example, each cutlery envelope contains a knife, fork and spoon as well as any suitable condiments, such as sachets of salt/pepper, and a napkin. Individual envelopes can be supplied to customers prior to dining. Each envelope can therefore contain all of the necessary cutlery and accessories that a customer may require when dining.

Use of a cutlery envelope also reduces the number of times cutlery is handled directly, for example by serving staff, and so offers the added benefit of increasing hygiene. Cutlery envelopes also offer protection from airborne transmission of pathogens from people in the vicinity of where cutlery is handled, stored or laid on a table, for example.

Nevertheless, it is to be appreciated that such cutlery envelopes do not eliminate the risk of contamination, particularly with regard to pathogens that may be prevalent in the catering establishment or which may have come into contact with the cutlery envelopes themselves during manufacture or prior to use.

Therefore, there exists a need to reduce the risk of transmission of pathogens to cutlery used in a catering environment.

### SUMMARY OF THE INVENTION

According to a first aspect of the invention there is provided a method of manufacturing a sanitised paper article comprising the steps: supplying a paper sheet having a first surface and a second surface to a flexographic printing apparatus having a first sanitising module; applying a sanitising solution to at least the first surface of the paper sheet using the first sanitising module, and forming an article from the sanitised paper sheet.

Certain aspects of the present invention provide a method of producing an article, for example an envelope, notepad or other such paper product, which reduces the risk of transmission of pathogens.

Certain aspects of the present invention provide a hygienic paper article, such as a hygienic cutlery envelope.

It will be appreciated that in certain embodiments, a paper article could be manufactured from multiple sheets of sanitised paper.

The flexographic printing apparatus may further comprises a second sanitising module and the method further comprises the step of applying a sanitising solution to the second surface of the paper sheet using the second sanitising module.

The step of applying a sanitising solution to the second surface may be subsequent to the step of applying a sanitising solution to the first surface.

The flexographic printing apparatus may further comprises at least one ink printing module and the method may further comprises the step of printing a graphic and/or text on at least one of the first and second surfaces of the paper sheet. The graphic and/or text may, for example, be printed on the first surface of the paper sheet.

The step of printing on at least one of the first and second surfaces of the paper sheet may be subsequent to the step of applying a sanitising solution to at least the first surface of the paper sheet.

The sanitising solution may comprise a sanitising agent and a carrier agent. The carrier agent may be a liquid. The sanitising agent may comprise ethanol. The sanitising solution may comprise at least 70% ethanol. The paper sheet is supplied from a continuous roll of paper.

The article may be an envelope.

The step of forming the envelope may comprise the steps of cutting a blank having a predetermined shape from the sanitised paper sheet, and configuring said blank to form a pocket having an aperture along one side.

The blank may comprise a back panel, a front panel and a closure panel arranged such that the back panel and the front panel can be folded towards each other to form the pocket and the closure panel can be folded over the aperture to inhibit access to the pocket.

The method may further comprise a step of applying an adhesive covered by a removable protective strip to the blank for securing the closure panel over the aperture.

The method may further comprise a step of forming a perforation extending across a region of the envelope such that a user can remove a portion of the envelope from the remaining envelope by tearing along the perforation.

The sanitising module may comprise a fluid supply unit, an application roller for applying a sanitising solution to a paper sheet and a transfer roller arranged to transfer the sanitising fluid from the fluid supply unit to the application roller.

The sanitising module may further comprise at least one doctor blade arranged to control the amount of sanitising solution transferred by the transfer roller to the application roller.

A sanitised paper article that has been manufactured in accordance with the method of any one of the preceding claims.

Various further features and aspects of the invention are defined in the claims.

Unless defined otherwise, all technical and scientific terms used herein have the meaning commonly understood by a person skilled in the art to which this invention belongs.

### BRIEF DESCRIPTION OF THE DRAWINGS

Embodiments of the present invention will now be described by way of example with reference to the accompanying drawings where like parts are provided with corresponding reference numerals and in which:

FIG. 1 is a schematic representation of a flexographic printing apparatus;

FIG. 2 is a flow chart illustrating process steps of a method manufacturing a sanitised paper article;

FIG. 3 is a blank used to form an envelope;

FIG. 4A shows a front side of an envelope;

FIG. 4B shows a rear side of an envelope;

FIG. 5A illustrates the correlation between cell densities and distribution;

FIG. 5B shows a comparison between dot sizes; and

FIG. 6 is table illustrating technical specifications for transfer rollers.

### DETAILED DESCRIPTION

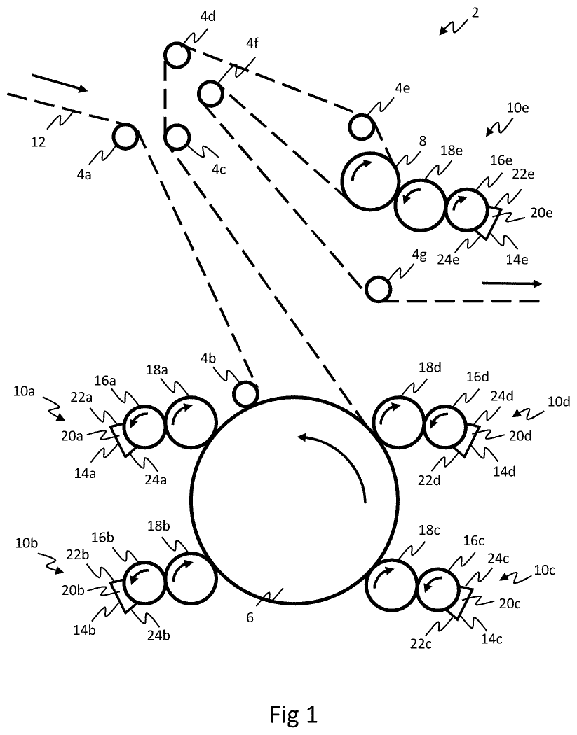

FIG. 1 shows a flexographic printing apparatus 2 comprising a plurality of guide rollers 4a, 4b, 4c, 4d, 4e, 4f, 4g, a first impression cylinder 6, a second impression cylinder 8, a first sanitising module 10a, first, second and third printing modules 10b, 10c, 10d, and a second sanitising module 10e.

First and second guide rollers 4a, 4b are arranged to guide a paper sheet 12 (which may also be termed a paper substrate) drawn from a roll (not shown) to an outer surface of the first impression cylinder 6 such that the paper sheet 12 is brought into contact with the first impression cylinder 6.

Second, third and fourth guide rollers 4c, 4d, 4e are arranged to guide the paper sheet 12 from the outer surface of the first impression cylinder 6 to the second impression cylinder 8 such that the paper sheet 12 is brought into contact with an outer surface of the second impression cylinder 8. Fifth and sixth guide rollers 4f, 4g are arranged to guide the paper sheet 12 away from the second impression cylinder 8 for further processing, such as cutting.

The printing modules 10b, 10c, 10d and sanitising modules 10a, 10e have the same construction. Each comprises a respective fluid (e.g. ink or sanitising solution) supply unit 14a, 14b, 14c, 14d, 14e, a respective transfer roller 16a, 16b, 16c, 16d, 16e and a respective application roller 18a, 18b, 18c, 18d, 18e.

Each fluid supply unit 14a, 14b, 14c, 14d, 14e comprises a fluid, and specifically a liquid, reservoir 20a, 20b, 20c, 20d, 20e formed, in part, by spaced apart first and second ‘doctor’ blades 22a, 22b, 22c, 22d, 22e and 24a, 24b, 24c, 24d, 24e. The doctor blades 22a, 22b, 22c, 22d, 22e, 24a, 24b, 24c, 24d, 24e are arranged to press against an outer surface of a respective transfer roller 16a, 16b, 16c, 16d, 16e so as to seal the reservoir 20a, 20b, 20c, 20d, 20e against the transfer roller 16a, 16b, 16c, 16d, 16e.

Each transfer roller 16a, 16b, 16c, 16d, 16e is a conventional transfer roller (which may also be known as a screen roller, coating roller or anilox roller) configured to collect a layer of liquid from a respective liquid supply unit 14a, 14b, 14c, 14d, 14e having a precise thickness. Each transfer roller 16a, 16b, 16c, 16d, 16e is a ceramic coated roller having a plurality of identical cells on its outer surface which have a predefined volume. The cells may be arranged in any suitable matrix, such as a honeycomb pattern. In the present embodiment each transfer roller 16a, 16b, 16c, 16d, 16e has a cell density of 160 cells/cm², but transfer rollers having cell densities between 100 cells/cm2 and 315 cells/cm² could be used (for applications requiring higher volumes of liquid transfer, lower cell densities are typically used, whereas for applications requiring lower volumes of liquid transfer, higher cell densities are typically used). Each cell is approximately 10cm deep and has a predetermined cell volume in the range 3.5 cm³ to 6 cm³, such as 5 cm³.

In general, the technical specification of a transfer roller determines the print quality. Typically, the objective is to use always the highest cell density which satisfy required colour densities or, where applicable, the required distribution of sanitising solution.

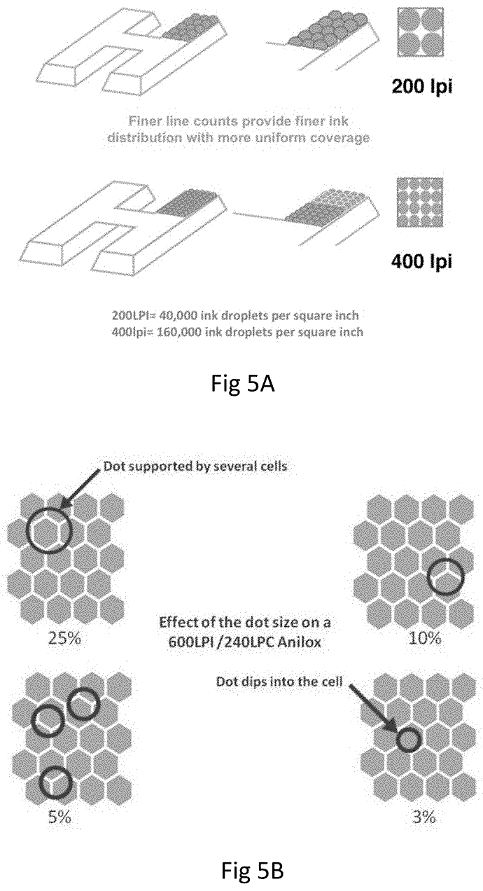

The higher the cell density (which correlates with a line count, as is known in the art of flexographic printing) the finer the ink/sanitising solution distribution, as illustrated by FIG. 5A. The cell density determines the total number of ink dots that will be transferred to an application roller. Higher cell densities place more cells, closer together. Smaller dots will dry faster producing less dot gain which will offer better image clarity or a more controlled application of sanitising solution.

When selecting a line count (i.e. cell density) for a process job, it is typical to specify a line count which is five times the desired printing resolution. This ratio allows the cell walls to adequately support the dot and keeps the dot from dipping into the cells, as illustrated by FIG. 5B.

Cell density is chosen in correlation with cell volume. For example, if printing solids (i.e. an unbroken image or continuous coating), such as the application of a sanitising solution, a volume of 8 cm³/m² is typically required. The cell density is therefore selected accordingly and so the line count could be approximately 400 LPI (which equates to 1600,000 cells per square inch).

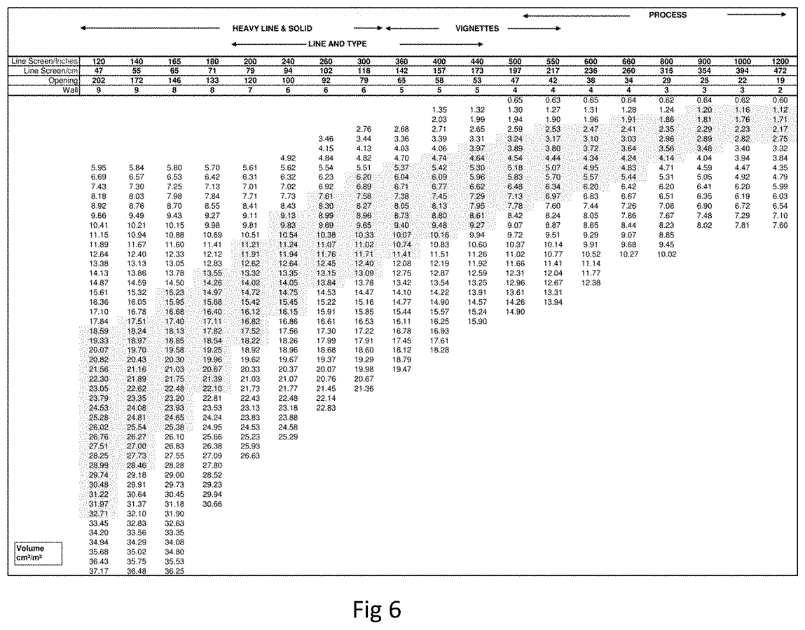

FIG. 6 provides a table illustrating example technical specifications for transfer rollers.

Each transfer roller 16a, 16b, 16c, 16d, 16e is arranged to contact a respective application roller 18a, 18b, 18c, 18d, 18e so as to deposit fluid on the application roller 18a, 18b, 18c, 18d, 18e.

Each application roller 18a, 18b, 18c, 18d, 18e is a conventional application roller (also termed a print roller) comprising a cylinder and a flexible plate (e.g. a printing plate or a coating plate) wrapped around the cylinder. In the embodiment shown, the flexible plates are photopolymer print plates. The plates have a shore hardness of approximately 40. Each flexible plate for the application rollers 18b, 18c, 18d of the printing modules 10b, 10c, 10d has portions which define an image (such as a logo) and/or text that is to be printed, as required. The text/images are created on the plates using a conventional process, such as by exposing portions of the plates defining the text/image to an ultraviolet light and then washing uncured regions of the surface away.

The first, second, third and fourth application rollers 18a, 18b, 18c, 18d are arranged circumferentially about the first impression cylinder 6 and spaced apart approximately equidistantly. Each application roller 18a, 18b, 18c, 18d is arranged for pressing engagement with the first impression cylinder 6 such that the flexible plate contacts the paper sheet 12 when the paper sheet 12 is wound around the first impression cylinder 6.

The fifth application roller 18e is arranged for pressing engagement with the second impression cylinder 8 such that the flexible plate contacts the paper sheet 12 when wound around the second impression cylinder 8.

The flexible plates of each of the first and fifth application rollers 18a, 18e are configured to apply a continuous coating of sanitising solution to the paper sheet 12.

Prior to operation, a paper sheet 12 is arranged to extend from a roll of paper (not shown) through the flexographic printing apparatus 2. The paper sheet 12 may be passed through an alignment station prior to reaching the printing apparatus 2 in order to align the paper for printing. The paper sheet 12 extends over the first guide roller 4a and down to the second guide roller 4b and the first impression cylinder 6, where it is brought into contact with an outer pressing surface of the first impression cylinder 6 as it passes between the second guide roller 4b and the first impression cylinder 6.

The paper sheet 12 extends circumferentially around the outer surface of the first impression cylinder 6 and passes between each of the application rollers 18a, 18b, 18c, 18d of the first sanitising module 10a and the first, second and third printing modules 10b, 10c, 10d, and the first impression cylinder 6.

The paper sheet 12 extends away from the outer surface of the first impression cylinder 6 and upwardly towards the third guide roller 4c where it passes around the third guide roller 4c before passing over the fourth guide roller 4d and down over the fifth guide roller 4e to the second impression cylinder 8.

The paper sheet 12 extends circumferentially around the outer surface of the second impression cylinder 8 and passes between the application roller 18e of the second sanitising module 10e and the second impression cylinder 8. The paper sheet 12 then extends upwardly and around the fifth guide roller 4f and then extend back towards and under the sixth roller 4f and away from the printing apparatus 2.

Each reservoir 20a, 20b, 20c, 20d, 20e is either filled with a liquid which is to be applied or else connected to such a liquid supply.

For example, the fluid reservoir 20a of the first sanitising module 10a is filled with a sanitising solution, such as a solution comprising at least 70% ethanol. The sanitising solution is clear in the sense that it has no discernible colour.

The fluid reservoir 20b of the first printing module 10b is filled with a first ink, such as an ink having a desired first colour.

The fluid reservoir 20c of the second printing module 10c is filled with a second ink, such as an ink having a desired second colour.

The fluid reservoir 20d of the third printing module 10d is filled with a third ink, such as an ink having a desired third colour.

The fluid reservoir 20e of the second sanitising module 10e is filled with a sanitising solution, such as a sanitising solution which is the same as the sanitising solution used in the first sanitising module 10a.

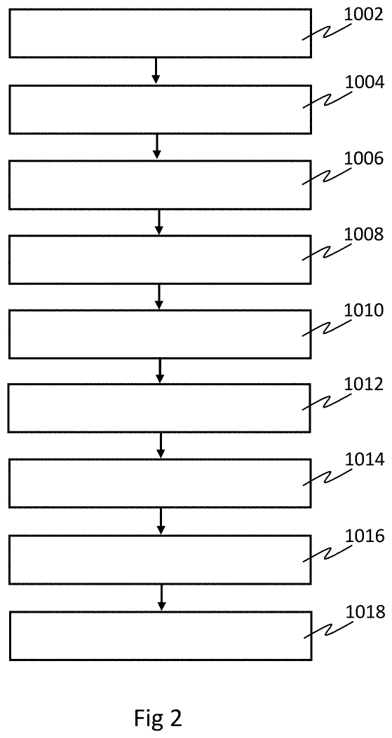

A flow diagram of a method of manufacturing a sanitised paper article, particularly a sanitised paper envelope, is shown in FIG. 2.

At step 1002, the first impression cylinder 6 is driven in an anti-clockwise direction (as viewed in FIG. 1) such that the paper sheet 12 is continuously drawn from the roll of paper over the first guide roller, between the second guide roller 4b and onto the outer surface of the first impression cylinder 6.

As the first impression cylinder 6 rotates, the paper sheet 12 passes between the application roller 18a of the first sanitising module 10a and the first impression cylinder 6.

At step 1004, the sanitising solution is applied by the application roller 18a onto a first surface of the paper sheet 12, which is the surface facing radially outwardly from the first impression cylinder 6. The sanitising solution is applied to the paper sheet 12 by first drawing the sanitising solution from the fluid reservoir 20a onto the transfer roller 16a and then depositing the sanitising solution onto the application roller 18a which, in turn, deposits the sanitising solution onto the paper sheet 12. The first doctor blade 24a ensures that the thickness of the sanitising solution on the transfer roller 16a is a substantially constant predetermined thickness. This is done by ‘scraping away’ excess sanitising solution not in the cells, and so ensures that the amount of sanitising solution transferred to the application roller 18a, and subsequently applied to the paper sheet 12, is a predetermined amount. Once the sanitising solution has been applied, the paper sheet 12 continues to travel with the first impression cylinder 6 towards the first printing module 10b where it passes between the application roller 18b of the first printing module 10b and the first impression cylinder 6.

At step 1006, the first ink is applied by the application roller 18b onto the first surface of the paper sheet 12 by drawing the first ink from the fluid reservoir 20b via the transfer roller 16b and transferring the first ink to the application roller 18b which is then deposited by the application roller 18b onto the paper sheet 12. Once the first ink has been applied, the paper sheet 12 continues to travel with the first impression cylinder 6 towards the second printing module 10c where it passes between the application roller 18c of the second printing module 10c and the first impression cylinder 6.

At step 1008, the second ink is applied by the application roller 18c onto the first surface of the paper sheet 12 by drawing the second ink from the fluid reservoir 20c via the transfer roller 16c and transferring the second ink to the application roller 18c which is then deposited by the application roller 18c onto the paper sheet 12. Once the second ink has been applied, the paper sheet 12 continues to travel with the first impression cylinder 6 towards the third printing module 10d where it passes between the application roller 18d of the third printing module 10d and the first impression cylinder 6.

At step 1010, the third ink is applied by the application roller 18d onto the first surface of the paper sheet 12 by drawing the third ink from the fluid reservoir 20d via the transfer roller 16d and transferring the third ink to the application roller 18d which is then deposited by the application roller 18d onto the paper sheet 12. Once the third ink has been applied, the paper sheet 12 leaves the surface of the first impression cylinder 6 and travels around the third guide roller 4c and over the fourth and fifth guide rollers 4d, 4e and onto the outer surface of the second impression cylinder 8.

At step 1012, the sanitising solution is applied by the application roller 18e onto the second surface of the paper sheet 12 by drawing the sanitising solution from the fluid reservoir 20e via the transfer roller 16e and transferring the sanitising solution to the application roller 18e which is then deposited by the application roller 18e onto the paper sheet 12. Once the sanitising solution has been applied to the second surface, the paper sheet 12 leaves the outer surface of the second impression cylinder 8 and travels around the sixth and seventh guide rollers 4f, 4g before continuing to a cutting process.

The second doctor blade 24b, 24c, 24d, 24e of each of the respective printing modules 10b, 10c, 10d and sanitising module 10e ensures that the thickness of ink or sanitising solution on the respective transfer rollers 16a, 16b, 16c, 16d, 16e is a substantially constant predetermined thickness.

The process sanitises both sides of a paper sheet. By applying sanitising solution first the sanitising solution completely coats the first surface before it is then printed upon. Applying the sanitising solution first also means that the sanitising solution is transferred to other parts of the flexographic printing apparatus 2, such as the guide rollers 4a, 4b, 4c, 4d, 4e, 4f, 4g and the other application rollers 18a, 18b, 18c, 18d, 18e which helps to continuously sanitise the entire apparatus 2.

The second impression cylinder 8, application rollers 18a, 18b, 18c, 18d, 18e, transfer rollers 16a, 16b, 16c, 16d, 16e and guide rollers 4a, 4b, 4c, 4d, 4e, 4f, 4g are driven directly or indirectly by the first impression cylinder 6.

Cutting and Preparation

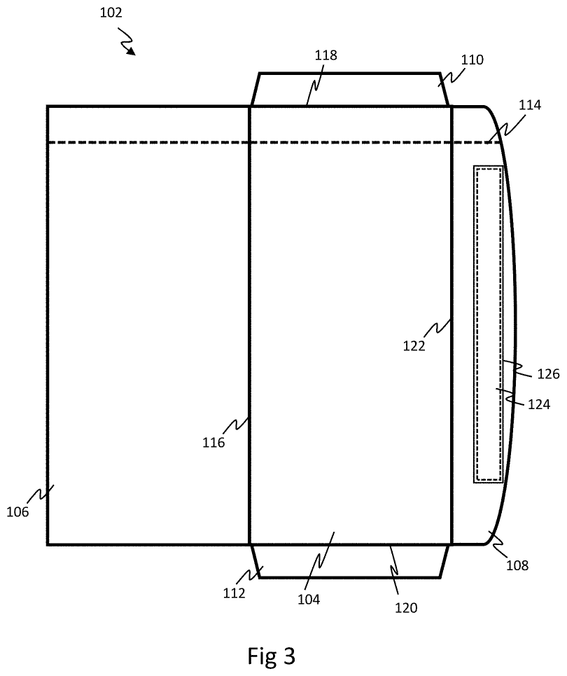

At step 1014, an envelope ‘blank’ 102 (see FIG. 3) is cut from the paper sheet 12 by a cutter (not shown). The blank 102 has a rectangular front panel 104, a rectangular back panel 106 and a closure panel 108. An upper tab 110 is provided at the top of the front panel 104 and a lower tab 112 is provided at the bottom of the front panel 104. A perforation 114 extends across an upper portion of the blank 102 and parallel to the upper edges of the front panel 104, back panel 106 and closure panel 108.

The back panel 106 is joined to the front panel 104 at a first fold line 116. The upper tab 110 is joined to the front panel 104 at a second fold line 118 and the lower tab 112 is joined to the front panel 104 at a third fold line 120. The closure panel 108 is joined to the front panel 104 at a fourth fold line 122.

At step 1016, a sealing adhesive 124 is applied along the closure panel 108 and covered by a removable protective strip 126. FIG. 3 shows the blank 102 arranged with the surface corresponding to the second surface of the paper sheet 12 from which it is cut facing upwardly.

Assembly

At step 1018, an envelope 202 is assembled from the blank 102 by folding the upper and lower tabs 110, 112 inwardly along the respective second and third fold lines 118, 120 towards the front panel 104. An adhesive is applied to the upwardly facing surface of the upper and lower tabs 110, 112. The back panel 106 is then folded inwardly along the first fold line 116 towards the front panel 104 and brought into contact with the upper and lower tabs 110, 112. The adhesive is then allowed to cure, if needed.

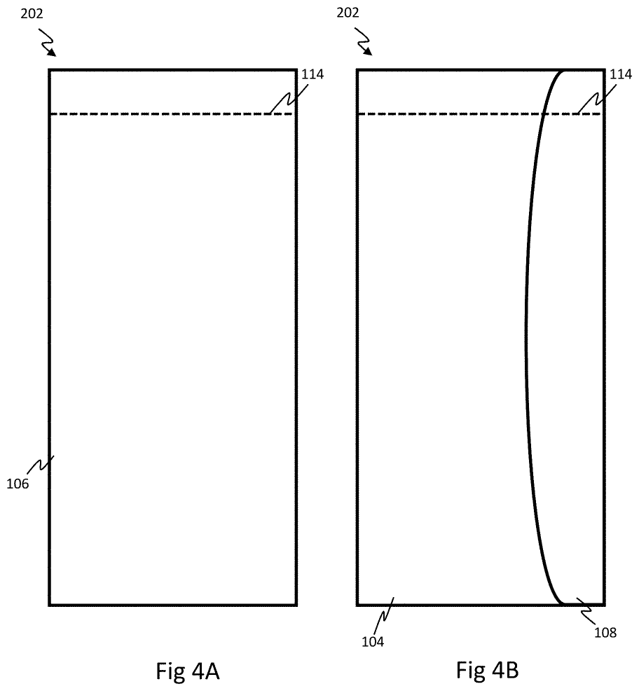

The finished envelope 202 has an inner surface, which corresponds to the second surface of the paper sheet 12, and an outer surface, which corresponds to the first surface of the paper sheet 12, which are sanitised. The assembled envelope 202 is shown from the front in FIG. 4A and from the back in FIG. 4B. The envelope 202 can then be packaged and transported for use.

The step of assembling the envelope 202 may be done mechanically by a machine. Assembly is carried out in a sanitised environment to avoid contamination of the envelope 202 prior to and during packaging for storage and transport. When assembled, the envelope 202 has a width of 110 mm and a length of 240 mm.

Use

The envelope 202 is a cutlery envelope for storing cutlery temporarily after it has been cleaned and prior to being needed by a user. The envelope 202 is intended for use in catering, such as a restaurant or café, in which customers are provided with cutlery by the caterer.

After cutlery has been cleaned (e.g. washed and dried) it is clean and typically sterile. It is immediately inserted into the envelope 202, for example by a member of the catering staff, suitably attired through an aperture defined by the front and back panels 104, 106 in the vicinity of the fourth fold line 122. Any condiments, such as sachets of salt/pepper, and accessories, such as a napkin, can also be inserted into the envelope 202.

The protective strip 126 is then removed to expose the adhesive 124 underneath and the closure panel 108 is folded inwardly along the fourth fold line 122 so that the closure panel 108 is brought into contact with the back panel 106. The adhesive 124 secures the two together with an ‘instant stick’ destructive bond. The cutlery can then be stored in the envelope 202 until being given to a customer.

On receipt of the envelope 202 containing the cutlery, a customer tears along the perforation 114 to remove the upper end portion of the envelope 202. The cutlery, and any condiments and accessories, can then be removed by the customer.

A benefit of the envelope 202 is that it reduces the risk of contamination of cutlery with pathogens during transport and storage after washing of the cutlery and before use by a customer. In particular, having a sanitised envelope also ensures that pathogens are not transferred to the cutlery by the envelope 202 itself.

It will appreciated that the method described above could be used to manufacture other types of envelopes, such as envelopes for containing medical pills, or other paper articles such as packaging, notebooks or sketchpads.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” or “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A method of manufacturing a sanitised paper article comprising the steps:
supplying a paper sheet having a first surface and a second surface to a flexographic printing apparatus having a first sanitising module;
applying a sanitising solution to at least the first surface of the paper sheet using the first sanitising module, and
forming an article from the sanitised paper sheet.

2. The method of claim 1, wherein the flexographic printing apparatus further comprises a second sanitising module and the method further comprises the step of applying a sanitising solution to the second surface of the paper sheet using the second sanitising module.

3. The method of claim 1, wherein the step of applying a sanitising solution to the second surface is subsequent to the step of applying a sanitising solution to the first surface.

4. The method of claim 1 wherein the flexographic printing
apparatus further comprises at least one ink printing module and the method further comprises the step of printing a graphic and/or text on at least one of the first and second surfaces of the paper sheet.

5. The method of claim 4, wherein the step of printing on at least one of the first and second surfaces of the paper sheet is subsequent to the step of applying a sanitising solution to at least the first surface of the paper sheet.

6. The method of claim 1, wherein the sanitising solution comprises a sanitising agent and a carrier agent.

7. The method of claim 6, wherein the carrier agent is a liquid.

8. The method of claim 6, wherein the sanitising agent comprises ethanol.

9. The method of claim 8, wherein the sanitising solution comprises at least 70% ethanol.

10. The method of claim 1, wherein the paper sheet is supplied from a continuous roll of paper.

11. The method of claim 1, wherein the article is an envelope.

12. The method of claim 11, wherein the step of forming the envelope comprises the steps of:
cutting a blank having a predetermined shape from the sanitised paper sheet, and configuring said blank to form a pocket having an aperture along one side.

13. The method of claim 12, wherein the blank comprises a back panel, a front panel and a closure panel arranged such that the back panel and the front panel can be folded towards each other to form the pocket and the closure panel can be folded over the aperture to inhibit access to the pocket.

14. The method of claim 13, further comprising a step of applying an adhesive covered by a removable protective strip to the blank for securing the closure panel over the aperture.

15. The method of claim 11, further comprising a step of forming a perforation extending across a region of the envelope such that a user can remove a portion of the envelope from the remaining envelope by tearing along the perforation.

16. The method of claim 1, wherein the sanitising module comprises a fluid supply unit, an application roller for applying a sanitising solution to a paper sheet and a transfer roller arranged to transfer the sanitising fluid from the fluid supply unit to the application roller.

17. The method of manufacturing of claim 16, wherein the sanitising module further comprises at least one doctor blade arranged to control the amount of sanitising solution transferred by the transfer roller to the application roller.

18. A sanitised paper article that has been manufactured in accordance with the method of claim 1.
